(19) United States
(12) Patent Application Publication
(10) Pub. No.: US 2004/0235755 A1
(43) Pub. Date: Nov. 25, 2004

(54) Use of amino acids amino acid alogues, sugar phosphates and sugar phosphate analogues for treatment of tumors, treatment of sepsis and immunosuppression

(76) Inventors: Erich Eigenbrodt, Linden (DE); Sybille Mazurek, Linden (DE); Helmut Grimm, Giessen (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/471,705

(22) PCT Filed: Jan. 17, 2002
(86) PCT No.: PCT/DE02/00212

(30) Foreign Application Priority Data
Mar. 13, 2001 (DE) 101 12 926.2

(51) Int. Cl. A61K 31/7024; A61K 31/401; A61K 31/198

(52) U.S. Cl. 514/23; 514/561; 514/423; 514/562

(57) ABSTRACT

The invention relates to methods for the treatment of tumors and/or for immune suppression and/or sepsis by modulating the association of the glycolysis enzyme complex/M2-PK and/or by inhibition of transaminases and/or separation of the binding of the malate dehydrogenase to p36 comprising administering a pharmaceutical composition comprising a substance selected from the group consisting of amino acids, amino acid analogs, sugar phosphates, sugar phosphate analogs, and mixtures of said substances.

### FIELD OF THE INVENTION

The invention relates to the use of sugar phosphates, sugar phosphate analogs, amino acids, and/or amino acid analogs for modulating metabolism processes.

### BACKGROUND OF THE INVENTION

Various diseases are caused by modifications in cellular metabolism. In particular in tumor tissue, the energy generation takes place at least partially via different mechanisms than in healthy tissue. These tumor-specific mechanisms are the starting points for tumor therapies, which specifically act on the tumor tissue and have comparatively few side effects. Therein the tumor growth is selectively inhibited and/or the apoptosis of tumor cells is initiated.

### PRIOR ART

It is known in the art that tumors are subject to a modified metabolism. This modified metabolism results in the use of glucose mainly used for nucleic acid synthesis. Simultaneously, a new energy source, the amino acid glutamine, is made accessible. Glutamine exists at high concentrations in all tissues. Typically, a tumor tissue is highly hypoxic, i.e. lacks sufficient oxygen, due to irregular vasculature in the tumor tissue. This makes clear that an adjustment to hypoxic conditions is a substantial factor in affecting tumor growth. The anaerobic reaction of glucose for the purpose of the energy generation by glycolysis is, therefore, a common feature of most tumour tissue aggregates. With regard to general, more detailed literature, reference is made to C. V. Dang et al., TIBS 24:68-72, 1999.

The pyruvate kinase (PK) is a key enzyme of glycolysis that catalyses the energy-supplying conversion of phosphoenolpyruvate into pyruvate. Four tissue-specific isoforms are known in the art, PK types L, R, M1 and M2 (see E. Eigenbrodt et al., Critical Reviews in Oncogenesis, Vol. 3, M. Perucho, Ed., CRC-Press, Boca Raton, Fla., pages 91-115, 1992). M2-PK is the embryonic form and replaces all other forms in proliferating cells and tumor cells (see G. E. J. Staal et al., Biochemical and Molecular Aspects of Selected Cancers, T. G. Pretlow et al., Eds., Academic Press Inc., San Diego, 1, pages 313-337, 1991, and U. Brinck et al., Virchows Archiv 424, pages 177-185, 1994). M2-PK protein of the rat consists of 530 amino acids and differs in only a single residue from human M2-PK (see T. Noguchi et al., J. Biol. Chem., 261, pages 13807-13812, 1986, and K. Tani et al., Gene, 73, pages 509-516, 1988). M2-PK is a glycolytic enzyme, which may exist in a highly active tetrameric form and also in a mildly active dimeric form. Only the highly active tetrameric form is associated in the glycolysis-enzyme complex.

The glycolysis-enzyme complex is an association of glycolysis enzymes, NDPK, adenylate kinase, RNA, A-raf and components of the protein kinase cascade. The transition between the two forms of the M2-PK regulates the glycolytic reaction in tumor cells (see Mazurek, S. et al., J. Cell. Physiol. (1996) 167:238-250; Mazurek, S. et al., Anticancer Res. (1998) 18:3275-3282; Mazurek, S. et al., J. Bioenerg. Biomembr., 29, pages 315-330, 1997). The activity of M2-PK thus controls the transition of the glycolytic pathway. If the M2-PK exists in the dimeric form, the glucose carbon atoms are fed to branching synthesis processes. If the M2-PK exists in the tetrameric form and as an associated form in the glycolysis-enzyme complex, the glucose is reacted very effectively under energy gain to pyruvate and lactate. The overexpression of M2-PK permits cells to survive under low oxygen conditions since PK does not need oxidative phosphorylation for the production of ATP. Generally, an increased amount of M2-PK is found in malignant tumours and in the blood of tumour patients.

The document Eigenbrodt, E. et al., Biochemical and Molecular Aspects of Selected Cancers, Vol. 2, p. 311 ff (1994), discloses the use of glucose analogs for inhibiting glycolysis. Another approach known in the art is the use of inhibitors of glycolytic isoenzymes, for instance by suitable complex formation or inhibition of complex formations. As a result the tumor cells are so to speak “starved out”. It is problematic for the above compounds that many of them are genotoxic and/or not sufficiently specific for tumor cells.

From the document Eigenbrodt et al. in Critical Reviews in Oncogenesis (1992) (Perucho, M. ed.) CRC-Press, Boca Raton, Fla., 3:91-115, it is known that fructose-1,6-bisphosphate leads to a displacement of the dimeric form to the highly active tetrameric form of M2-PK, thus teaching that the glycolytic flux in tumor cells is controllable. From said document it is further known that alanine and leucine inhibit M2-PK.

The document, U. Mangold et al., Eur. J. Biochem. 266:1-9 (1999) discloses that 2-cyano-3-hydroxy-but-2-(4-trifluoromethyl-phenyl)-amide (in the following CHBA) affects glycolysis and also discloses a new active ingredient for treating inflammatory illnesses and autoimmune reactions.

Transaminases are enzymes that transfer, during transamination, amino groups from 2-amino acids to 2-keto acids. They are a sub-group of transferases. The prosthetic group is pyridoxal phosphate. An inhibition of transaminases leads to an increase in amino acids. From the document E. Eigenbrodt et al., Biochemical and Molecular Aspects of Selected Cancers, Vol. 2, p. 311 ff (1994), it is known in the art that aminooxyacetate and cycloserine inhibit glutamate pyruvate transaminase and can inhibit the proliferation of cells.

### TECHNICAL OBJECT OF THE INVENTION

The invention is based on the technical object of providing active agents, which are capable of inhibiting the proliferation of cancer cells and thus the growth of neoplastic tumors. It is also an object of the invention to provide agents capable of suppressing defensive over-reactions of the body, such as septic shock, autoimmune diseases, transplant rejections as well as acute and chronic inflammatory diseases, with only to little or no cytotoxicity to normal cells of the blood, of the immune system and the tissue cells.

### BASICS OF THE INVENTION

For achieving said technical object, the invention teaches the use of a substance selected from the group consisting of amino acids, amino acid analogs, sugar phosphates, sugar phosphate analogs and mixtures of said substances for producing a pharmaceutical composition for treating tumors and/or for immune suppression and/or sepsis by modulating the association of the glycolysis enzyme complex/M2-PK and/or by inhibiting transaminases and/or separating the binding of the (mitochondrial) malate dehydrogenase to p36.

The invention is first of all based on the finding that in tumor cells, the ratio of tetrameric to dimeric M2-PK is approximately 50:50. Subsequently, it has been found that a modification of this ratio, i.e. a displacement to one of the two forms, is suitable for tumor therapy. It has been found that with complete tetramerization of the M2-PK, nucleic acid synthesis and consequently, cell proliferation, is inhibited. In the case of complete dimerisation, there is, however, an inhibition of the energy gain from glucose with the consequence of apoptosis, an equally positive therapeutic effect. Surprisingly, both effects can thus be used for tumor therapy. Cytotoxic effects are not to be expected, since this metabolic condition is specific to tumor tissue.

In addition to modifications in the pyruvate kinase isoenzyme structure, tumor generation results in a disappearance of the NAD dependent cytosolic glycerol 3-phosphate dehydrogenase. This causes hydrogen to be transported from the glycolytic glycerin aldehyde 3-phosphate dehydrogenase reaction via the malate aspartate shuttle into the mitochondria. This in turn leads to the activation of the decomposition reaction of glutamine into pyruvate and lactate (glutaminolysis). Glutaminolysis secures the pyruvate and energy provision under conditions wherein the M2-PK is inactivated. An important component of the malate aspartate shuttle is the pre-stage mitochondrial malate dehydrogenase which is bound to phosphoprotein p36 in the cytosol The binding of the mitochondrial malate dehydrogenase to p36 in the cytosol can be terminated by amino acids, by sugar phosphates as well as analogs thereof.

It has further been discovered that a modulation of the association glycolysis enzyme complex/M2-PK may also take place indirectly, i.e. without direct binding of an active ingredient to M2-PK. If, for example, the transamination is inhibited, and/or the binding of the malate dehydrogenase to p36 is removed, this will in turn lead to an increase or decrease in amino acids, which in turn will interact with M2-PK and consequently modulate the association.

In addition to glutamate pyruvate transaminase, glutamate oxalacetate transaminase, glutamate 3-hydroxypyruvate transaminase and other branched-chain α-keto carbonic acid transaminases can be inhibited by the compositions and active agents of the present invention.

The term analogs as used herein designates compounds that can be derived from the structures of natural amino acids or sugars, i.e. different therefrom, but able to effect the same or an even stronger modulation of the glycolysis enzyme complex/M2-PK association, transaminase inhibition and/or removal of the p36-malate dehydrogenase binding than the basic natural substance. An analog may in particular be a derivative, i.e. another non-naturally occurring group may replace a naturally occurring functional group or an H atom. This applies to side chains as well as to the main structure; for example a cyanide group may in particular replace the carboxyl group of an amino acid. In the case of sugar phosphate analogs, a cyanide group may replace one or more phosphate groups. Amino acid analogs are also the forerunners of amino acids, α-keto acids, and in particular α-keto acids wherein a cyanide (—CN) group replaces the —COOH group.

### PREFERRED EMBODIMENTS OF THE INVENTION

Various non-limiting embodiments of the invention are possible. For instance, a pharmaceutical composition according to the invention may contain several compounds used according to the invention. Further, a pharmaceutical composition according to the invention may contain an active ingredient different from an active ingredient used according to the invention in the form of a combination preparation. The various active ingredients may be prepared in a single dosage form, i.e. the active ingredients are mixed in the dosage form. It is however also possible to prepare the various active ingredients in spatially separated dosage forms of identical or different type.

With regard to the active ingredient used according to the invention it is possible that the substance is selected from the group consisting of serine, cycloserine, valine, leucine, isoleucine, proline, methionine, cysteine, amino isobutyrate, aminooxyacetate, CHBA, fructose-1,6-bisphosphate, glycerate-2,3-bisphosphate, glycerate-3-phosphate, ribose-1,5-bisphosphate, ribulose-1,5-bisphosphate, analogues of such compounds and mixtures of such substances.

Preferably the substance is selected from the group consisting of compounds of the formula I and mixtures of such compounds.

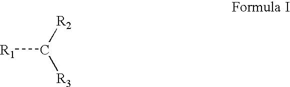

wherein R1=-NR4R5 or an amino acid residue, if applicable derivatized, R2=-COOH, —CN or —NR4R5, with R4 and R5 being identical or different and being H, C1-C18 alkyl, aryl or aralkyl, if applicable substituted with -J, —Cl and/or —F, R3==O.

These particularly preferred substances are typically 2 or α-oxonitriles or keto acids (if applicable, esterified). These substances are amino acid analogs of high efficiency.

It has to be noted, with regard to cycloalkyl and aryl groups, that homo as well as heteroatomic aromatic groups are within the scope of the invention. Examples of heterocyclic groups are: furanyl, thiophenyl, pyrrolyl, isopyrrolyl, 3-isopyrrolyl, pyrazolyl, 2-isomidazolyl, triazolyl, oxazolyl, isooxzolyl, thiazolyl, isothiazolyl, pyridyl, pyrazinyl, pyrimidinyl, pyridazinyl, piperazinyl, triazinyl, oxazinyl, indenyl, benzofuranyl, benzothiofuranyl, indolyl, isoindazolyl, benzoxazolyl, and the mentioned groups may be in part hydrated. Examples of such compounds are provided as follows:

As counter ions for ionic compounds according to formula I can be used Na⁺, K⁺, Li⁺ or cyclohexylammonium.

The drugs produced with the compounds according to the invention may be administered in an oral, intramuscular, periarticular, intraarticular, intravenous, intraperitoneal, subcutaneous or rectal manner. Particularly preferred, however, is intravenous administration, in particular in the administration of CHBA or aminooxyacetate (NH₂—CO—COOH) or sugar phosphates or sugar phosphate analogs.

The invention also relates to a method for preparing a drug which is characterized by at least one compound used according to the invention, which is mixed with a pharmaceutically suitable and physiologically well tolerated carrier and also, if applicable, with further suitable active ingredients, additional or auxiliary substances and prepared to a desired dosage form.

Suitable solid or liquid galenic dosage forms include, for example, granulates, powders, dragees, tablets, (micro) capsules, suppositories, syrups, juices, suspensions, emulsions, drops or injectable solutions as well as preparations with sustained release of the active ingredient. These dosage forms are prepared using standard techniques and materials, such as carrier substances, explosion, binding, coating, swelling, sliding or lubricating agents, flavoring substances, sweeteners and solution mediators.

Auxiliary substances that may be used include, for example, magnesium carbonate, titanium dioxide, lactose, mannite and other sugars, talcum, milk protein, gelatine, starch, cellulose and its derivatives, animal and plant oils such as cod-liver oil, sunflower, peanut or sesame oil, polyethylene glycols and solvents, such as sterile water and mono or poly-valent alcohols, e.g. glycerin.

Preferably the drugs are prepared and administered in dosage units, each unit containing as an active component a defined dose of the compound according to formula I of the invention. With solid dosage units such as tablets, capsules, dragees or suppositories, this dose may be I to 5,000 mg, preferably 50 to 1,000 mg, and for injection solutions in an ampoule form 1 to 5,000 mg, preferably 50 to 2,000 mg for intramuscular injection, or 1 to 100 mMol, preferably 10 to 100 mMol for intraperitoneal injection. For intravenous applications corresponding doses can be used, reduced by a factor of 0.5 to 0.1.

For treating an adult patient weighing from 50 to 100 kg for example 70 kg, daily doses of 20 to 5,000 mg active ingredient, preferably 500 to 3,000 mg, are indicated. Under certain circumstances, higher or lower daily doses may be advisable. The administration of the daily dose may be a one-time administration in the form of a single dosage unit or several smaller dosage units as well as multi-administration of separate doses in certain intervals.

In the following, the invention is explained in more detail with reference to examples representing embodiments only.

### EXAMPLE 1

### Tumour Model

As a tumor model, immuno-competent adult rats were used, treated with IV infusion. This animal model provides better correlation to human therapy than immuno-incompetent nude mice, which are commonly used. The animal tests were approved according to paragraph 8 section 1 of the German Animal Protection Act, and were performed according to the recommendations of the Tieraerztliche Vereinigung fuer Tierschutz e.V. (Veterinarians' Association for Animal Protection). Male inbreed rats (Sprague-Dawley, 200-250 g, Charles River, Sulzfeld, Germany) were used as tumor recipients.

Novikoff hepatoma was used as the source of tumor cells. Of several tested, experimentally produced tumors, the Novikoff hepatoma best all requirements of a solid tumor having all signs of malignity and similarity to hepatocellular carcinoma in humans. The Novikoff hepatoma was induced by Alex B. Novikoff in 1951 by feeding a diet containing 0.06 % 4-dimethylazobenzene (butter yellow) to female Sprague-Dawley rats (Novikoff B., A transplantable rat liver tumour induced by 4-dimethylaminoazobenzene. Cancer Res. 1951; 17:1010). The growth of this liver tumor as an ascites tumor as well as a solid tumor displays typical malignity characteristics such as hyperchromatism, polymorphism, increased mitosis rate and nucleus-plasma relation displaced in favor of the nucleus. The chromatin structure in the tumor cells appears in an irregular form, and the nuclei are indented, round and oval.,

The Deutsches Krebsforschungsinstitut in Heidelberg provided the Novikoff hepatoma cells. The cells were received in Hank's solution and intraperitoneally injected in a sterile manner into a Sprague-Dawley rat for passaging. Within a week, approximately 50 ml hemorrhagic ascites were generated and removed in a sterile manner. The pellet generated after centrifugation at 1,300 rpm for five minutes in a Falcon tube was prepped for further purification of cells from other ascites components by washing with 50 ml Dulbecco's MEM (Gibco BRL, Eggenstein) and centrifugation at 1,300 rpm for five minutes. The supernatant was decanted, and the pellet was mixed in Dulbecco's +40% fetal calf serum (FCS). 0.7 ml cell suspension and 0.7 freezing medium each were filled in Nunc tubes, air-tight sealed, pre-cooled for five minutes at −20° C. and for 12 hours at −80° C., and then deep-frozen in liquid nitrogen. The freezing medium was 40% Dulbecco's, 40% FCS and 20% DMSO.

The cells were prepared for the application as follows: after thawing, the pellet was reacted in a Falcon tube with 50 ml of a medium (Dulbecco's +40% FCS), pre-heated to 37° C., and centrifuged for five minutes at 1,300 rpm. The supernatant was removed, and the process was repeated.

After centrifugation and decantation of the supernatant, the pellet was suspended in HBSS, 100 microlitres were sampled with an Eppendorf pipette and counted for determining the number of vital cells after vital staining with erythrosine (BioMed, Munich, Germany) in a Neubauer counting chamber. The cell suspension was diluted after centrifugation and decantation with HBSS until the suspension contained 5×10⁶ vital cells per ml. 1 ml of this suspension was received in an insulin syringe and subcutaneously injected into the back of the rat.

For this purpose, the animal was anesthetized with ether, a skin fold was shaved and disinfected with 70% alcohol and a cannula No. 14 was inserted in the longitudinal direction from caudal to cranial, and the tumor cells were subcutaneously injected.

### EXAMPLE 2

### Treatment

The infusion of the test animals with substances according to the invention started as soon as the tumor had a volume of 1 ml. The tumor size was determined by CT-supported volumetry. For this purpose, the rats were intramuscularly paralyzed with 0.315 mg fentanyl citrate/kg body weight (Hypnorm®, Janssen, Beersee, Belgium). By means of a Somatom Plus 4-scanner (Siemens, Erlangen, Germany), a spiral CT with a layer thickness of 2 mm, a pitch of 1.5 and 2 mm increment at 120 kVp with 320 mAs was performed. A soft tissue algorithm was employed.

In one rat to be treated, a silicone tube (SilasticR 0.012 inch by 0.025 inch, No. 602-105 HH 061999, Dow Corning Corp., Midland, Mich., USA) was pushed by means of chloroform on the end of a 5 cm long spiral-shaped piece of PE 10 (polyethylene) catheter (Clay Adams, Parsippany, N.J., USA). The opposite end was connected to a 30 cm long piece of PE 20 catheter. The silicone piece was introduced into the left jugular vein of the recipient and secured with a ligature, as previously described [Weeks J R. Long term intravenous infusion, In: Meyers RD (ed.) Methods in Psychobiology, Academic Press 1972;2:155]. The spiral-shaped catheter portion reached the subcutaneous tissue and provided for the necessary extra length, in order to prevent catheter dislocation in the case of head movements of the animal. The other end was guided to the outside through the skin, protected in a metal spiral hose fixed by means of a girdle to the animal, and connected to an infusion pump permitting a body weight-adapted continuous infusion. The infusions took place while the animals were in a metabolic cage.

Ten randomized animals per group were each administered a substance (1.25 mM aminooxyacetate or 10 μM CHBA) over the course of 10 days, beginning with a tumor volume of 1 ml, Control animals received an isovolumic amount of NaCl. All animals had free access to water and R3-EWOS-ALAB stock food (ALAB, Sollentuna, Sweden). After 10 days, the animals were intramuscularly paralyzed with 0.315 mg fentanyl citrate/kg body weight (hynormR, Janssen, Beersee, Belgium), the tumor was removed, and its volume was determined by water displacement techniques.

### EXAMPLE 3

### Results

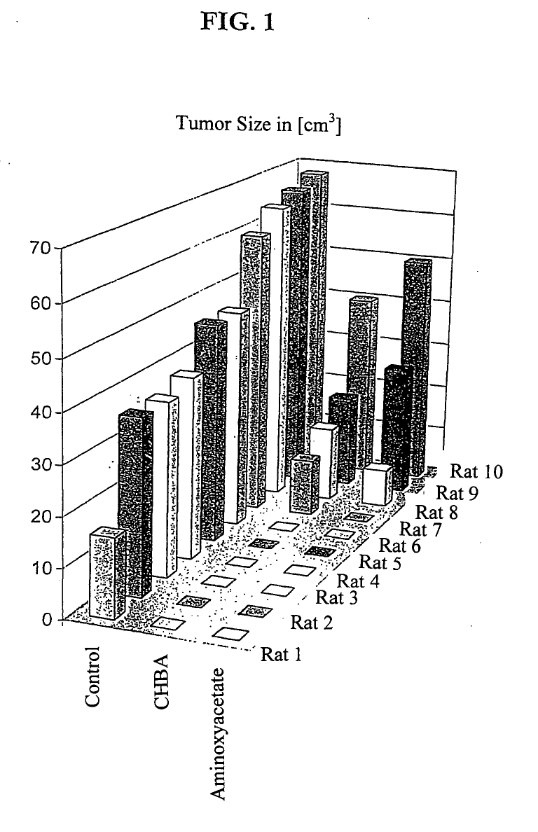

FIG. 1 shows the results obtained. Whereas the control animals had tumors of considerable size, a substantial inhibition of the tumor growth was observed in CHBA or aminooxyacetate administered animals. If the tumor was relatively small at the beginning of the treatment, a practically complete inhibition of tumor growth, and in some cases, apoptosis, was observed.

### EXAMPLE 4

### Dose-Dependence of Proliferation Inhibition from the Dose

In this example, the dependency dose of proliferation inhibition for various compounds according to the invention is shown.

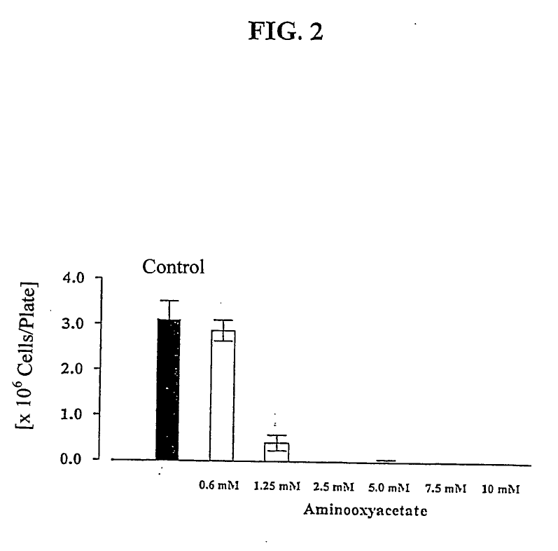

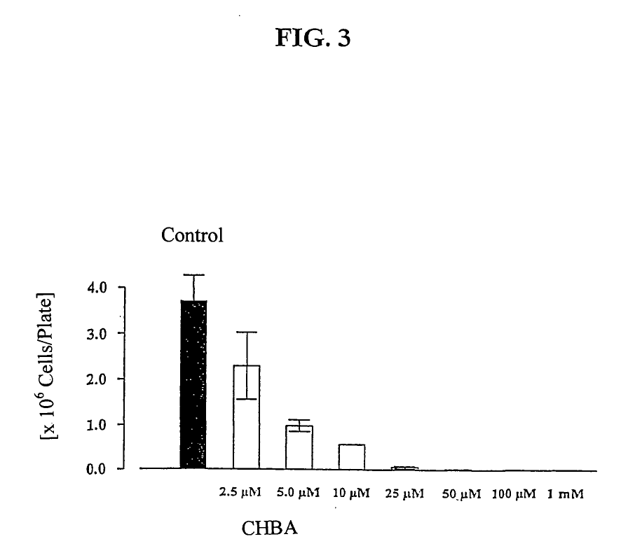

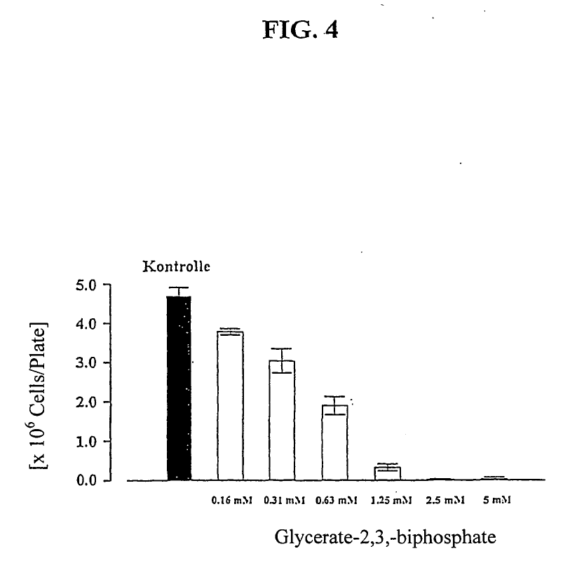

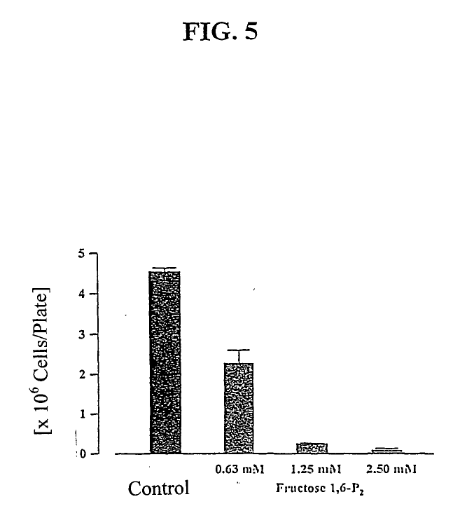

For the experiments, Novikoff hepatoma cells were cultivated in a conventional manner. The control substance contained a solvent without an active ingredient. The other groups received different doses of a respective test compound. After four days of cultivation with or without active ingredient, the cell density was determined using standard methods. In FIG. 2 is shown the dose-dependence of obtained cell densities for aminooxyacetate, in FIG. 3 for CHBA, in FIG. 4 for glycerate-2,3-bisphosphate, and in FIG. 5 for fructose-1,6-bisphosphate. In all cases, a practically complete inhibition was observed, at higher dosage levels.

1. A method for the treatment of tumors and/or for immune suppression and/or sepsis by modulating the association of the glycolysis enzyme complex/m2-pk and/or by inhibition of transaminases and/or separation of the binding of the malate dehydrogenase to p36 comprising administering a pharmaceutical composition comprising a substance selected from the group consisting of amino acids, amino acid analogs, sugar phosphates, sugar phosphate analogs, and mixtures of said substances

2. The method of claim 1, wherein the substance is selected from the group consisting of serine, cycloserine, valine, leucine, isoleucine, proline, methionine, cysteine, amino isobutyrate, aminooxyacetate, CHBA, fructose-1,6-bisphosphate, glycerate-2,3-bisphosphate, glycerate-3-phosphate, ribose-1,5-bisphosphate, ribulose-1,5-bisphosphate, analogues of such substances and mixtures of such substances.

3. The method of claim 1, wherein the substance is selected from the group consisting of compounds of the formula I and of mixtures of such compounds, 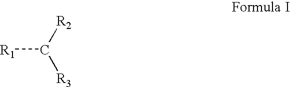
wherein R1=-NR4R5 or an amino acid residue, if applicable derivatized,
wherein R2=-COOH, —CN or —NR4R5,
wherein R4 and R5 are identical or different and are H, C1-C18 alkyl, aryl or aralkyl, if applicable substituted with -J, —Cl and/or —F, and
wherein R3==O.

4. The method of claims 1 to 3, wherein the pharmaceutical composition is prepared for an intravenous application.

5. The method of claims 1 to 3, wherein the pharmaceutical composition is prepared for an administration of a daily dose of 0.1 to 80 mg per kg body weight.

6. The method of claim 4, wherein the pharmaceutical composition is prepared for an administration of a daily dose of 0.1 to 80 mg per kg body weight.
